(19) United States
(12) Plant Patent Application Publication
(10) Pub. No.: US 2004/0068776 P1
(43) Pub. Date: Apr. 8, 2004

(54) Anthemis tinctoria plant named 'Charme'

(75) Inventor: Brian Kabbes, Suameer (NL)

Correspondence Address: BURNS DOANE SWECKER & MATHIS L L P, POST OFFICE BOX 1404, ALEXANDRIA, VA, 22313-1404, US

(73) Assignee: INSPIRATION PLANT CV, Lisse (NL)

(21) Appl. No.: 10/680,183

(22) Filed: Oct. 8, 2003

Related U.S. Application Data
(63) Continuation of application No. 10/152,807, filed on May 23, 2002, now abandoned.

(30) Foreign Application Priority Data
Feb. 14, 2002 (NL) ATM 1

(51) Int. Cl. A01H 5/00

(52) U.S. Cl. PLT/263

(57) ABSTRACT

A new and distinct Anthemis tinctoria cultivar is provided that is the result of a controlled breeding program. The plant displays a distinctive dwarf growth habit. Attractive intense golden yellow blossoms are formed in abundance on a substantially continuous basis from June to October. The foliage contrasts well with the golden yellow blossom coloration. Non-viable seeds are formed. The new cultivar is well suited for growing as attractive ornamentation in pots or in the landscape.

### BOTANICAL/COMMERCIAL CLASSIFICATION

Anthemis Tinctoria/Anthemis

### VARIETAL DENOMINATION

cv. ‘Charme’

### SUMMARY OF THE INVENTION

The new cultivar of Anthemis Tinctoria was created by artificial pollination wherein two parents crossed which previously had been studied in the hope that they would contribute the desired characteristics. Plants of this species sometimes are identified as Golden Marguerite. The breeding program that created the new cultivar took place during 1993 to 1998 at Suameer, The Netherlands, with the first cross being made during July 1993. The object of the breeding program was to create a distinctive dwarf-type Anthemis plant that displays a long flowering season combined with reduced fertility.

The female parent (i.e., the seed parent) was formed by the crossing of Anthemis tinctoria, ssp. fussii and Anthemis tinctoria, cv. ‘KK94/2’ (non-patented in the United States). The male parent (i.e., the pollen parent) was formed by the crossing of Anthemis tinctoria, cv. ‘KK96/17’ (non-patented in the Untied States and Anthemis tinctoria ssp. australis (non-patented in the United States). The seeds resulting from the cross were sown and small plants were obtained which were physically and biologically different from each other. Selective study resulted in the identification of a single plant of the new cultivar of the present invention.

It was found that the new Anthemis plant of the present invention displays:

(a) a distinctive dwarf growth habit

(b) forms in abundance on a substantially continuous basis attractive intense golden yellow ligulate blossoms

(c) forms non-viable seeds, and

(d) is well suited for growing as attractive ornamentation in pots or in the landscape.

The new cultivar well meets the needs of the horticultural industry in view of its distinctive combination of characteristics. Previously know Anthemis plants form longer stems and are significantly taller overall plants. The dwarf stature of the new cultivar renders it to be particularly well suited for growing as a distinctive border or patio plant.

The new cultivar was first asexually reproduced by the use of cuttings on Mar. 12, 1998 at Suameer, The Netherlands. It has been demonstrated that characteristics of the new cultivar are firmly fixed and are retained through successive generations of asexual propagation.

The new cultivar of the present invention has been named ‘Charme’. ‘Charme’ has not been observed under all possible environmental conditions to date. Accordingly, it is possible that the phenotype may vary somewhat with variations in the environment, such as temperature, light, day length, etc.

### BRIEF DESCRIPTION OF THE PHOTOGRAPH

The accompanying photographs depict typical plants of the new cultivar while growing at Suameer, The Netherlands.

FIG. 1 shows a typical potted plant of the new ‘Charme’ cultivar wherein an abundance of intense golden yellow blossoms and dark green foliage are displayed.

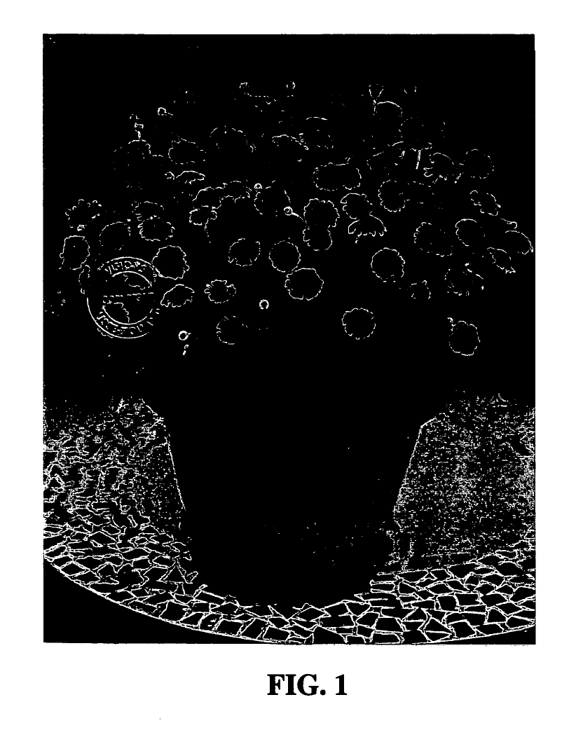

FIG. 2 shows a closer view of blossoms and dark green foliage of the new ‘Charme’ cultivar.

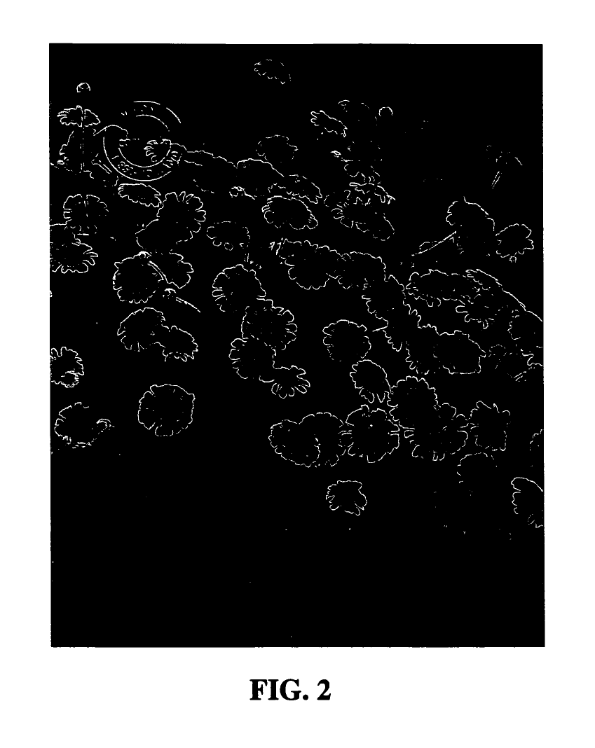

### DETAILED DESCRIPTION

The ‘Charme’ plants were reproduced from cuttings and were grown pots at Suameer, The Netherlands. The color chart used in the identification of color is the R.H.S. Colour Chart of The Royal Horticultural Society, London.

Growth habit: Fast-growing dwarf perennial. A twelve week-old plant commonly displays a height of approximately 40 cm and a width of approximately 50 cm. As the plant further matures, there is an increasing width and little change in height. Previously known Anthemis plants commonly display a height at least approximately twice that of the new ‘Charme’ cultivar.

Foliage: Two-pinnasect with oblong to linear segments, and lanate on the under surface. On August 6th the leaves were Green Group 137C in coloration. The foliage possesses a fragrance typical of Anthemis when crushed, and contrasts nicely with blossom coloration.

Inflorescence: Forms in abundance attractive daisy-shaped ligulate blossoms. On August 6th the flower diameter ranged from approximately 3.4 to 4.9 cm. The ligules were Canary Yellow, Yellow Group 9A. The flowering is substantially continuous from June to October with good reblooming. During 1999 the first flower appeared on June 11th, and last flower faded on October 18th.

Fertility: Some seeds are formed; however, no viable seeds have been observed during observations to date.

Disease/Pest resistance: No special susceptibility to diseases and insects have been observed.

Drought/Heat resistance: Good resistance to drought and heat has been displayed during observations to date.

Culture: Performs best in well-drained soil.

Hardiness: Can be grown in U.S.D.A. Hardiness Zone Nos. 3 to 9.

Usage: Provides attractive rich-flowering ornamentation when grown as a border plant or when potted and displayed on a patio.

I claim:

1. A new and distinct cultivar of Anthemis tinctoria plant is provided that displays:
(a) a distinctive dwarf growth habit,
(b) forms in abundance on a substantially continuous basis attractive intense golden yellow ligulate blossoms,
(c) forms non-viable seeds, and
(d) is well suited for growing as attractive ornamentation in pots or in the landscape;
substantially as illustrated and described.
